(19) United States
(12) Patent Application Publication
(10) Pub. No.: US 2025/0208088 A1
(43) Pub. Date: Jun. 26, 2025

(54) COMPACT SCALABLE DISSOLVED OXYGEN SENSOR

(71) Applicant: Yale University, New Haven, CT (US)

(72) Inventors: MOHSEN NAMI, New Haven, CT (US); DANIEL BACHESCHI, New Haven, CT (US); SONYA SAWTELLE, New Haven, CT (US); MARK REED, New Haven, CT (US)

(21) Appl. No.: 18/852,059

(22) PCT Filed: Mar. 20, 2023
(86) PCT No.: PCT/US2023/064692

§ 371 Date: Sep. 27, 2024

Related U.S. Application Data
(60) Provisional application No. 63/325,793, filed on Mar. 31, 2022.

(51) Int. Cl. G01N 27/404; G01N 27/407

(52) U.S. Cl. CPC G01N 27/404; G01N 27/4075

(57) ABSTRACT

An oxygen sensor device comprises a Clark-type sensor electrode comprising thin-film electrode leads overlaid with a solid-state proton conductive matrix (PCM) wherein at least one dimension of the electrode is less than 25 μm. An oxygen sensor system comprises an oxygen sensor device, a sensing window above the electrode, at least one contact pad, and at least one lead line electrically connecting the at least one contact pad to the oxygen sensor electrode. Methods of oxygen sensing are also disclosed.

### CROSS-REFERENCE TO RELATED APPLICATIONS

This application claims priority to U.S. provisional application No. 63/325,793 filed on Mar. 31, 2022, incorporated herein by reference in its entirety.

### BACKGROUND OF THE INVENTION

Throughout ecological, biomedical, and chemical applications, the close monitoring of dissolved oxygen levels may prove critical during process control, environmental condition monitoring, and clinical diagnostics. In industry, they are useful for quality control and assurance in water production as well as sewage management (see Holenda, 2008). For scientific applications in aquaculture, they are widely deployed to closely monitor aquatic systems that support various forms of aquatic life (see Hargreaves, 2002). In clinical applications where reliability, accuracy, and biofunctionality are paramount, suitable dissolved oxygen sensors have proved immensely beneficial.

One such clinical application is neurological operations for traumatic brain injuries (TBI). Moderate to severe traumatic brain injury (TBI) is a critical public health and socio-economic problem worldwide. It is a major cause of mortality among young adults and lifelong disability for its survivors. There are more than 5 million people in the US with TBI related disabilities, and the incidence of TBI worldwide is rising. TBI pathophysiology is divided into two phases: the initial neuronal injury (or primary injury) followed by secondary injury. In caring for severely brain injured patients, being able to minimize secondary injury is crucial.

Damage to living brain tissue caused by an external mechanical force necessitates invasive forms of surgery during which the close monitoring of blood oxygen levels within brain tissue is paramount for preventing tissue hypoxia (see Chue et al., 2015). Subsequent forms of “secondary injuries” that may develop as a consequence of TBI such as ischemia, hypoxia, seizures, and fevers, also have a direct impact on patient recoveries from various forms of cerebral injury. As such, it is critical to monitor patients' cerebral status via parameters such as intracranial pressure, blood flow, and brain tissue oxygen. Compact, simple, and minimally invasive dissolved oxygen sensors are necessary for brain tissue health monitoring in order to prevent secondary brain injury.

Multimodality monitoring allows neurointensivists the ability to monitor multiple physiologic parameters simultaneously, which provides critical insight into brain ischemia, hypoxia, and seizures, the major drivers of secondary injury. The management of severe TBI in the neuro-intensive care unit is moving away from a pure “threshold-based” treatment approach toward consideration of patient specific characteristics. Multimodality cerebral monitoring is critical to understanding patient specific characteristics, thus allowing one to tailor interventions on an individualized level.

Current approaches to TBI monitoring employ multiple invasive probes. Multiple separate devices cause a number of challenges, including increased risk of infection from multiple entry points, multiple electronic units from different vendors with different software systems, and different IT and biomedical engineering staff training requirements.

Chemical sensing modalities such as titration and iodimetry measure dissolved oxygen levels with high accuracy but require long processing times and delicate experimental setups (see Xiao et al., 2020). Optical methods for oxygen detection such as photochemical fluorescence often require complex and costly experimental setups and technologies. In addition, photochemical probes are frequently unsuitable for minimally invasive medical applications due to their high rigidity and resulting lack of maneuverability (see Chue et al., 2015). Finally, the use of lab instrumentation such as mass spectrometry and gas chromatography systems present an accuracy-cost tradeoff with long processing times and the inability to perform real-time measurements. Electrochemical sensing, on the other hand, offers a simple, powerful sensing modality with the possibility of real-time monitoring. In addition, the electrochemical modality is easily implemented at the micro-scale via Complementary Metal Oxide Semiconductor (CMOS) fabrication technologies, inheriting a mature manufacturing paradigm from the semiconductor industry. For these reasons, electrochemical sensing has emerged as a leading modality for the accurate, reliable, and low-cost monitoring of brain tissue oxygen levels.

The Clark-type electrode is a popular electrochemical dissolved oxygen sensor design that presents a tri-electrode system covered by a semi-permeable membrane that exploits reduction-oxidation reactions to generate dissolved oxygen readings. Biased at least equal to or less than the electrochemical reduction potential of oxygen and hydrogen peroxide with respect to the reference electrode, the working electrode provides the reaction site for the reduction of dissolved oxygen with the surrounding water molecules (see Kim, 2000). The reference electrode serves to stabilize the electrochemical system in solution (see Li, 2009; McLaughlin, 2005).

Thus, there is a need in the art for improved systems and methods of dissolved oxygen sensing.

### SUMMARY OF THE INVENTION

Some embodiments of the invention disclosed herein are set forth below, and any combination of these embodiments (or portions thereof) may be made to define another embodiment.

In one aspect, an oxygen sensor device comprises a Clark-type sensor electrode comprising thin-film electrode leads overlaid with a solid-state proton conductive matrix (PCM) wherein at least one dimension of the electrode is less than 25 μm.

In one embodiment, the electrode comprises an ultramicroelectrode. In one embodiment, the Clark-type sensor electrode includes a working electrode. In one embodiment, the Clark-type sensor electrode includes a counter electrode. In one embodiment, the Clark-type sensor electrode includes a reference electrode. In one embodiment, the device further comprises titanium/gold, titanium/platinum, chromium/gold, nickel/gold or silver/silver-chloride electrodes overlaid with a PCM constructed from Nafion. In one embodiment, the electrode comprises a serrated geometry. In one embodiment, the electrode comprises an omega geometry. In one embodiment, the electrode comprises a basic geometry. In one embodiment, wherein the electrode comprises an interdigitated (IDE) geometry. In one embodiment, the device is configured to measure dissolved oxygen concentration. In one embodiment, the device is configured to measure brain tissue oxygen (P_{BT}O₂).

In another aspect, an oxygen sensor system comprises an oxygen sensor device as described above, a sensing window above the electrode, at least one contact pad, and at least one lead line electrically connecting the at least one contact pad to the oxygen sensor electrode.

In one embodiment, the system further comprises a protective layer overlaying the PCM. In one embodiment, the protective layer comprises polydimethylsiloxane (PDMS), Polyetheretherketone (PEEK), Polytetrafluoroethylene (PTFE), Polypropylene (PP), Polystyrene (PS), Polyurethane, Polycarbonate (PC), Polyethylene, terephthalate (PET), Polymethyl methacrylate (PMMA), polymide, or Parylene-C. In one embodiment, the system further comprises an adhesion promoting compound. In one embodiment, the adhesion promoting compound comprises polyvinylpyrollidone (PVP) or SU-8. In one embodiment, the system further comprises a substrate. In one embodiment, the substrate comprises Kapton, glass, ceramic, silicon, or dielectric on silicon.

In another aspect, an oxygen measurement method comprises providing an oxygen sensor including an electrode, applying a voltage equal to or less than the electrochemical reduction potential of oxygen and hydrogen peroxide to the electrode, conducting a polarographic voltage sweep, generating a sensing current of 1 nano-amp or larger, and calculating dissolved oxygen concentration based on the sensing current.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1A through FIG. 1D show exemplary Clark electrode geometries (101, 102, 104) for exemplary dissolved oxygen sensors 100 in accordance with some embodiments. FIG. 1A shows a serrated electrode design 101 featuring a sawtooth-like counter electrode layout. FIG. 1B shows an omega electrode design 102 due to its working electrode design. FIG. 1C shows a basic electrode design 103 featuring a simpler rectangular working electrode design. FIG. 1D shows an inter-digitated electrode (IDE) electrode design 104 due to its inter-digitated electrodes.

FIG. 2 shows the working principles of a Clark sensor geometry for dissolved oxygen electrochemical sensing. The working electrode is biased below the reference electrode by at least the reduction potentials of oxygen and hydrogen peroxide in order to reduce dissolved oxygen and induce electronic charge transport. Oxygen concentration is then captured by measuring the current flowing at the working electrode.

FIG. 3A through FIG. 3D show experimental details for an exemplary dissolved oxygen sensor 100 in accordance with some embodiments. FIG. 3A shows a CAD drawing of a standard overall electrochemical sensor geometry, featuring the sensing window 105 and electrode contact pads 106. FIG. 3B shows an experimental setup, featuring a YSI ProDo Optical Probe in solution alongside the dissolved oxygen sensor. The container also had input ports for Nitrogen and Oxygen gas. FIG. 3C shows a sensor chip with metal leads attached for forming the biasing and sensing circuit with a Semiconductor Parameter Analyzer (SPA). FIG. 3D shows the overall electrical, signal, and gas paths throughout the experimental setup.

FIG. 4A through FIG. 4D are plots showing exemplary experimental results in accordance with some embodiments. FIG. 4A is a plot showing polarographic I-V trace for an exemplary interdigitated electrode (IDE) geometry 104 sensor device 100 with a 20 μm width and 10 μm separation, featuring multiple response signals across a voltage sweep from 0V to −1V at four chosen dissolved oxygen concentrations: 8.3 mmHg, 25.1 mmHg, 41.6 mmHg, and 58.6 mmHg. FIG. 4B is a plot showing signal currents for the exemplary device 100 at the four different dissolved oxygen concentrations under a −0.7V device bias. FIG. 4C is a plot of the exemplary device 100 current response according to a 50.3 mmHg change in oxygen concentration plotted against working electrode area. A clear correlation between working electrode area is present for the devices possessing standard microelectrode dimensions (red symbols), whereas ultramicroelectrode devices (blue symbols) appear to lose the correlation. FIG. 4D is a plot showing the calculated effective permeabilities of the PDMS/Nafion membrane plotted against the exemplary device 100 signal response currents (μA), displaying a clear positive correlation between signal response and effective permeability. The effective permeability is defined as a combination of vertical penetration of the O2 gas into the permeable layer and lateral diffusion at steady state.

FIG. 5A through FIG. 5C are plots showing analytical computations of isoconcentration lines as electrode dimensions approach ultramicroelectrode scales. FIG. 5A shows the isoconcentration lines for an electrode possessing a critical dimension of 3 mm. FIG. 5B shows the isoconcentration lines for an electrode possessing a critical dimension of 300 μm. FIG. 5C shows the isoconcentration lines for an electrode possessing a critical dimension of 20 μm, illustrating the expansion of ultramicroelectrodes' diffusion regions far beyond their borders.

FIG. 6 is a table showing exemplary device 100 signal response data resulting from a 50.3 mmHg change in dissolved oxygen gas concentration in solution in accordance with some embodiments. The working electrode areas and geometry specifications for each device are also presented. Furthermore, the table shows a comparison between micro-electrode geometries and ultramicroelectrode geometries.

### DETAILED DESCRIPTION OF THE INVENTION

It is to be understood that the figures and descriptions of the present invention have been simplified to illustrate elements that are relevant for a clearer comprehension of the present invention, while eliminating, for the purpose of clarity, many other elements found in systems and methods of dissolved oxygen sensing. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein is a dissolved oxygen sensor.

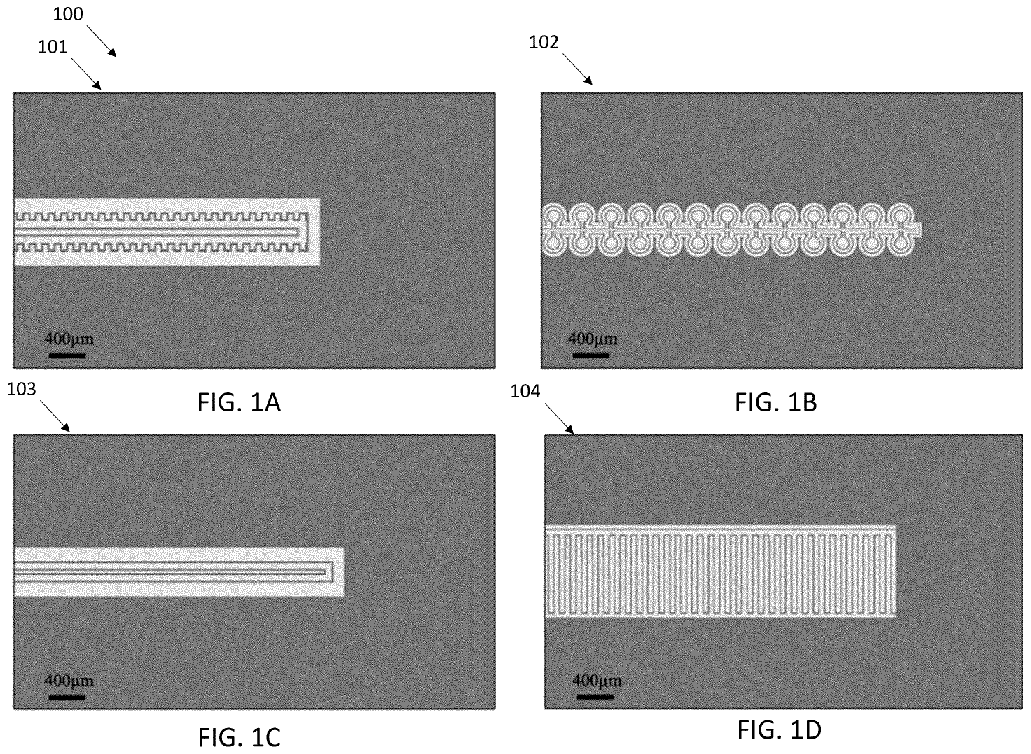

FIG. 1A through FIG. 1D show exemplary Clark electrode geometries (101, 102, 103, 104). In some embodiments, each electrode geometry (101, 102, 103, 104) includes a working electrode, a counter electrode, and/or a reference electrode. Clark-style electrochemical sensors detect dissolved oxygen without consuming the oxygen itself by driving the reduction of oxygen at the working electrode while driving its production at the counter electrode according to the following redox reaction equations:

O₂+2H⁺+2e⁻→H₂O₂(Working Electrode) (1)

H₂O₂+2H⁺+2e⁻→2H₂O(Working Electrode) (2)

2H₂O→4H⁺+O₂+4e⁻(Counter Electrode) (3)

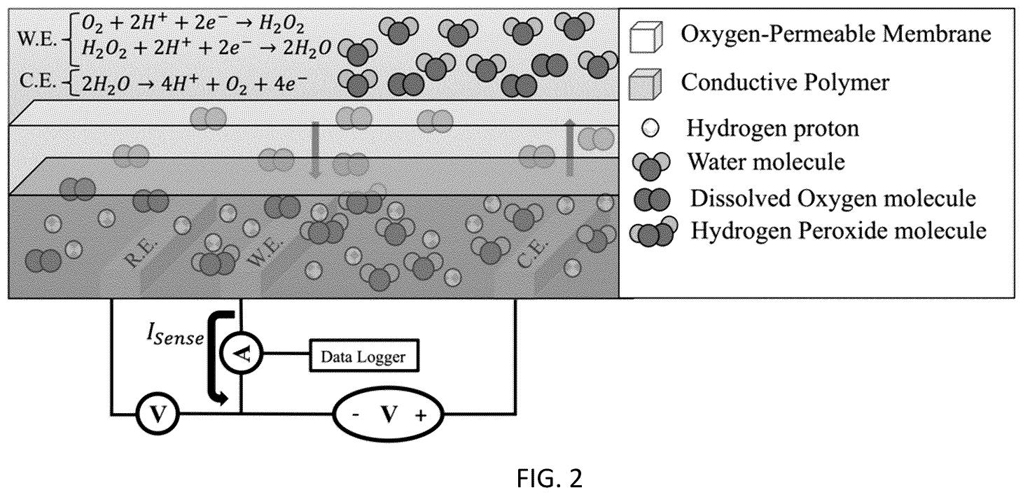

The oxygen is reduced and then reacts with hydrogen protons to yield hydrogen peroxide, which in turn is reduced to produce two water molecules (FIG. 2). Oxygen is produced at the counter electrode along with 4 electrons and 4 hydrogen ions. Upon oxygen production the counter electrode accepts electrons, thereby returning them to the sensing circuit. The working electrode gives up four electrons during the reduction of oxygen and hydrogen peroxide, generating a current through the working electrode that is proportional to the concentration of dissolved oxygen. The resulting current is then described by the following equation:

$${I}_{sense} = \frac{{nP}_{effective} {FA}_{s}}{{t}_{m}} {P}_{{O}_{2}} \qquad ( 4 )$$

where n is the number of electrons exchanged at the working electrode, P_{effective} is the effective permeability of the selective membrane, F is Faraday's Constant, Aₛ is the surface area of the working electrode, tₘ is the thickness of the selective membrane, P_{O}_{₂} is the oxygen's partial pressure in solution (Chue et al., 2015). With a strong linear dependence on permeability and electrode surface area, the electrode sensing configuration's performance necessitates electrode design optimization. An analytical solution for the differential sensing current with respect to partial oxygen pressure can be defined by (Kim et al., 2000):

$$\frac{\partial {I}_{sense}}{\partial {P}_{{O}_{2}}} = ( \frac{{nFA}_{s}}{{t}_{m}} ) {P}_{effective} \qquad ( 5 )$$

Electrochemical dissolved oxygen sensors employing sensing schemes depend significantly on the mass transport of molecular oxygen through the semi-permeable membrane and conductive polymer in order to generate a signal current proportional to the oxygen concentration. As a result, the protective, selective membrane isolating the sensing region must be permeable to dissolved oxygen. With this under consideration, it becomes clear that the diffusive mechanics of the movement of dissolved oxygen towards the working electrode are paramount for sensor operation. Such sensors effectively operate in one of two regimes, depending on the electrochemical properties of the sensing system: the kinetic and diffusion-limited regimes.

When biased at low voltages, the sensor operates in the kinetic regime where the current response is highly dependent on the bias voltage applied across the counter and working electrodes (McLaughlin et al., 2002; Wiranto et al., 2018). After a transition voltage point, the rate of oxygen reduction at the working electrode matches the oxygen repletion rate via diffusion; from that point on, the sensor operates in the diffusion-limited regime (McLaughlin et al., 2002; Wiranto et al., 2018). As a result, the sensor current becomes linearly dependent on the concentration of dissolved oxygen in solution. The point of transition from the kinetic regime to the diffusion-limited regime is determined by the ratio of the diffusion-limited current to the sensing current at electrochemical equilibrium, with smaller ratios leading to larger kinetic regime bias ranges (McLaughlin, 2001; McLaughlin et al., 2002).

The dissolved oxygen sensor's versatility may be greatly expanded by tuning the membrane's properties to better suit specific environments. Membrane biocompatibility, durability, and permeability may all be modulated to adapt the electrochemical sensor towards particular applications.

In some embodiments, thin-film electrode leads are overlaid with a solid-state proton conductive matrix (PCM). In some embodiments, the oxygen sensor 100 comprises electrodes made of a combination of two or more of the following metals: titanium, chromium, nickel, platinum, silver/silver chloride or gold, overlaid with a PCM constructed from Nafion or a combination of Nafion with micro or nanoparticles. In some embodiments, a protective layer that is permeable to oxygen, such as polydimethylsiloxane (PDMS), Polyetheretherketone (PEEK), Polytetrafluoroethylene (PTFE), Polypropylene (PP), Polystyrene (PS), Polyurethane, Polycarbonate (PC), Polyethylene, terephthalate (PET), Polymethyl methacrylate (PMMA), polymide, or Parylene-C, overlays the PCM to increase membrane stability. In some embodiments, the oxygen sensor 100 is configured to measure brain tissue oxygen (P_{BT}O₂). In some embodiments, the oxygen sensor 100 is configured to measure intracranial oxygen (icO). In some embodiments, the oxygen sensor 100 is operated in cyclic voltammetry (CV) mode. In some embodiments, the performance of the oxygen sensor 100 can be improved with the use of polyvinylpyrollidone (PVP) or SU-8 as an adhesion promoting compound. In some embodiments, the performance of the oxygen sensor 100 can be improved with the use of Kapton, glass, ceramic, silicon, or dielectric on silicon, as the substrate.

In some embodiments, the width of the electrode is about 1 μm to 200 μm, about 5 μm to 100 μm, about 20 μm to 40 μm, about 20 μm, less than about 25 μm, less than about 24 μam, less than about 23 μm, less than about 22 μm, less than about 21 μm, less than about 20 μm, or any other suitable size. In some embodiments, the electrode is an ultramicroelectrode. In some embodiments the electrode separation is about 0.5 μm to 100 μm, about 5 μm to 50 μm, about 10 μm to 30 μm, or any other suitable size.

One exemplary practical application of the dissolved oxygen sensor 100 described herein is as an oxygen sensor in a sensor probe, such as the multiplexed implantable sensor probe described in U.S. patent application Ser. No. 15/975,067, which is hereby incorporated herein by reference in its entirety. Due to its small size, the oxygen sensor 100 can be utilized in many aspects such as, for example, ecological, biomedical, and chemical applications, where the close monitoring of dissolved oxygen levels can prove critical during process control, environmental conditions monitoring, and clinical diagnostics, as well as in the treatment and monitoring of various cerebral traumas.

### Experimental Examples

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore specifically point out exemplary embodiments of the present invention and are not to be construed as limiting in any way the remainder of the disclosure.

In order to arrive at an optimum microelectrode design, four different electrode topologies were investigated (FIG. 1A through FIG. 1D). Variations of the widely used interdigitated electrode (IDE) 104 as well as a circular electrode geometry were tested, and their respective performances measured with changes of dissolved oxygen concentration in solution.

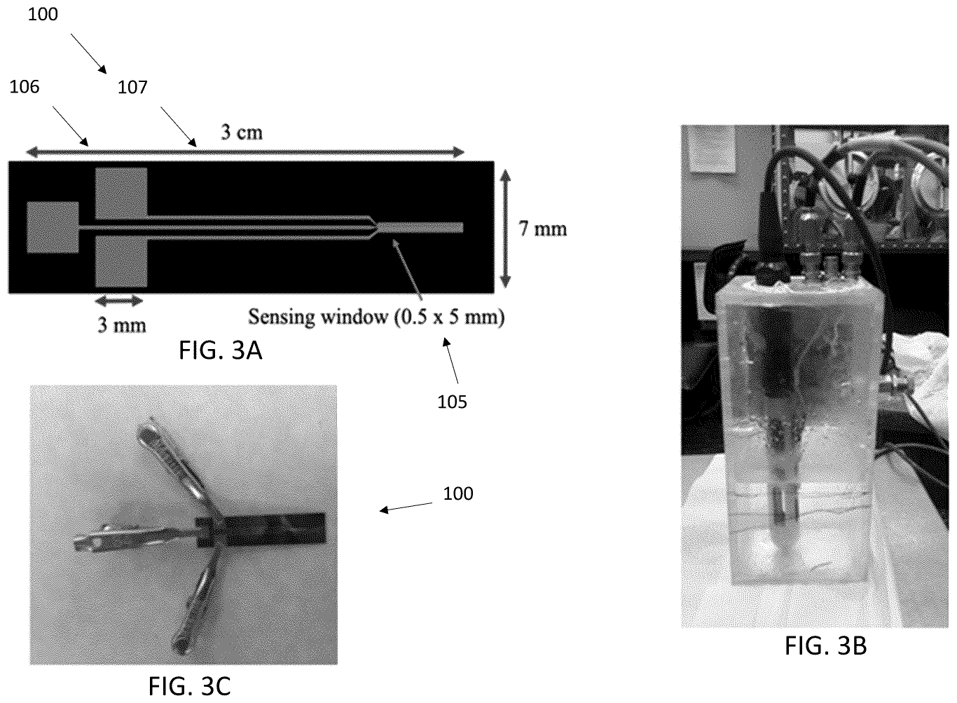

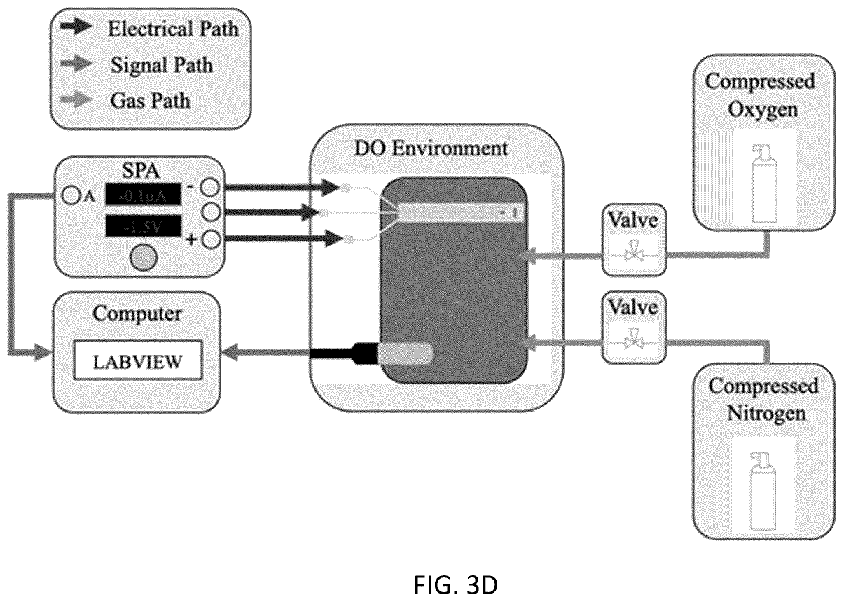

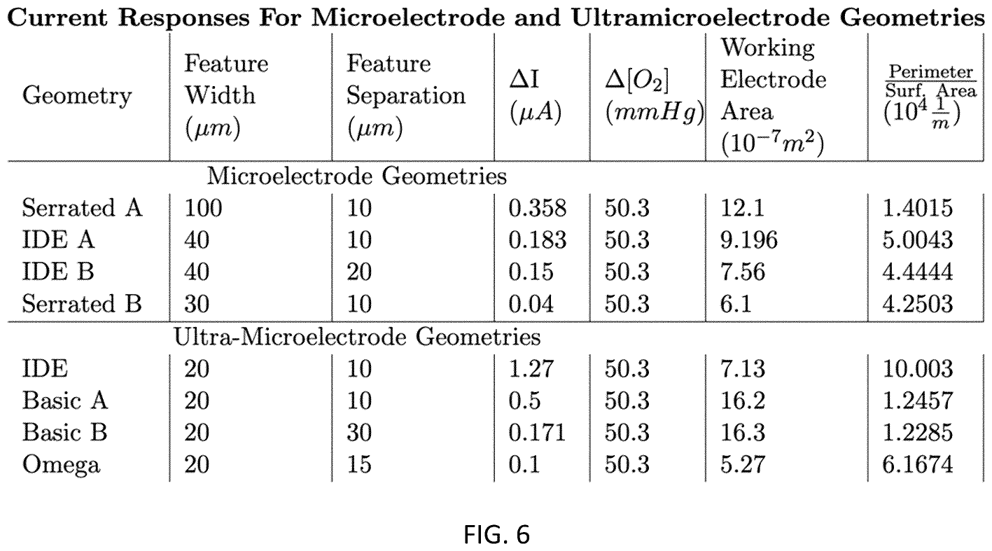

Exemplary experimental oxygen sensor devices 100 were fabricated in a cleanroom facility. The devices 100 were grown on a silicon wafer with 2 μm of thermal oxide on top, to mitigate for potential electrode leakage paths. Electrodes were patterned via standard photolithography techniques in the cleanroom using Shipley S1818 photoresist. The electrodes (101, 102, 103, 104) were metallized with 10 nm of titanium covered by 100 nm of gold. The layout for each electrochemical sensor was standardized, with a 9 mm² gold contact 106 for each electrode, a 2.5 mm² sensing window 105 containing the electrodes (101, 102, 103, 104), and the resulting lead lines 107 from the window to the contacts (FIG. 3A). The variations in electrode geometry took place in the sensing window 105 according to the pre-determined topologies (101, 102, 103, 104). A total of eight geometric variations (FIG. 6) were created by adjusting the width and spacing of certain features present in each of the four general electrode designs (101, 102, 103, 104).

The oxygen-permeable, biocompatible Nafion membrane over the electrodes (101, 102, 103, 104) was formed via a dip-coating protocol resulting in thicknesses on the order of 500 nm-600 nm. An additional PDMS layer, which is permeable to oxygen, and ranging from 7 μm to 121 μm in thickness, was coated over the Nafion membrane in order to better protect the delicate Nafion from solution and thereby increase sensor longevity.

The electrochemical sensors 100 were interfaced with directly via a Semiconductor Parameter Analyzer (SPA) machine (Agilent 4156B), which served as both the voltage bias source for driving oxygen reduction as well as an ammeter for detecting sensing currents. All three of the sensor's 100 electrode pads 106 were connected to the SPA via metal leads (FIG. 3C). A container was filled with deionized water rich in dissolved oxygen, and both the electrochemical sensor's electrodes and a commercial YSI ProDo Optical Probe were immersed in the water (FIG. 3B). The ProDo Probe generated control measurements for the oxygen concentrations against which to evaluate the accuracy of the electrochemical sensor. The SPA was used to conduct polarographic sweeps from 0V to −1V across the working-counter electrodes, with the counter electrode grounded along with the reference electrode. Polarographic sweeps were captured at four different oxygen concentrations: 8.3 mmHg, 25.1 mmHg, 41.6 mmHg, and 58.6 mmHg. Oxygen concentration was reduced by bubbling nitrogen gas into the water, with the nitrogen displacing the dissolved oxygen. A voltage sweep was conducted once the system reached steady state after each step decrease in oxygen concentration. Sensing current data from the SPA and the oxygen concentration data from the ProDo Probe were captured by a computer running Labview for visual recording of the data. FIG. 3D illustrates the overall experimental setup, with connections from the SPA driving the reduction-production reactions, the oxygen concentration control circuit, as well as the signal data paths.

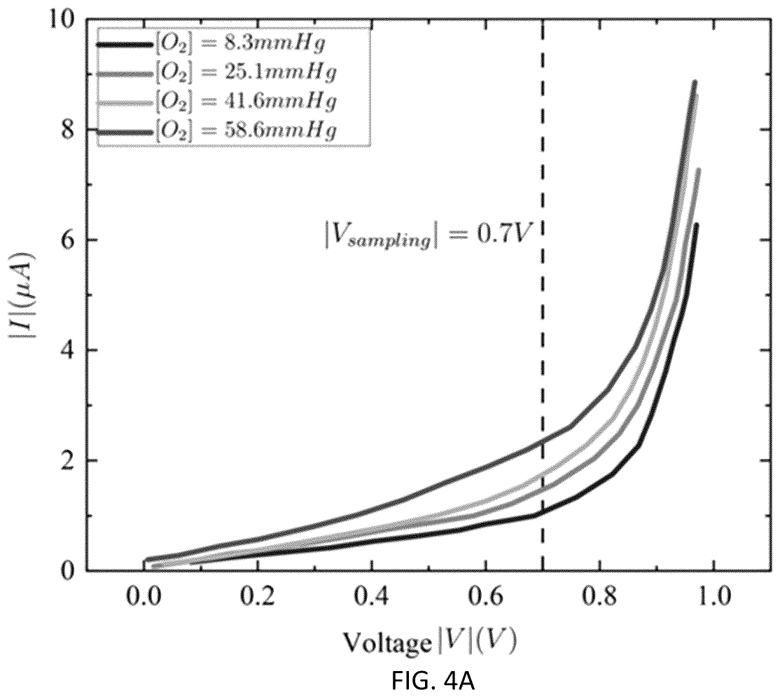

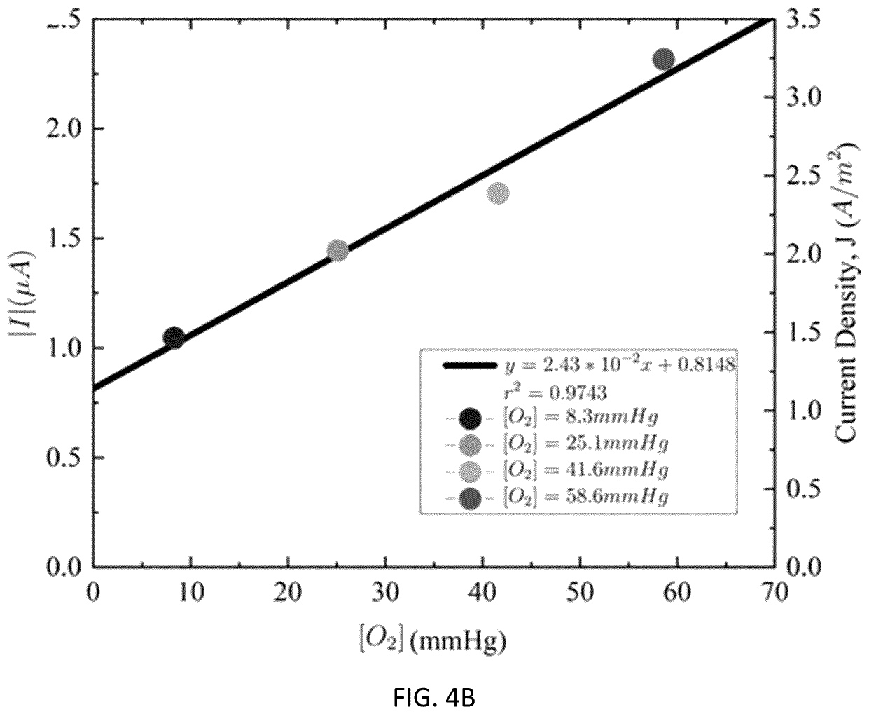

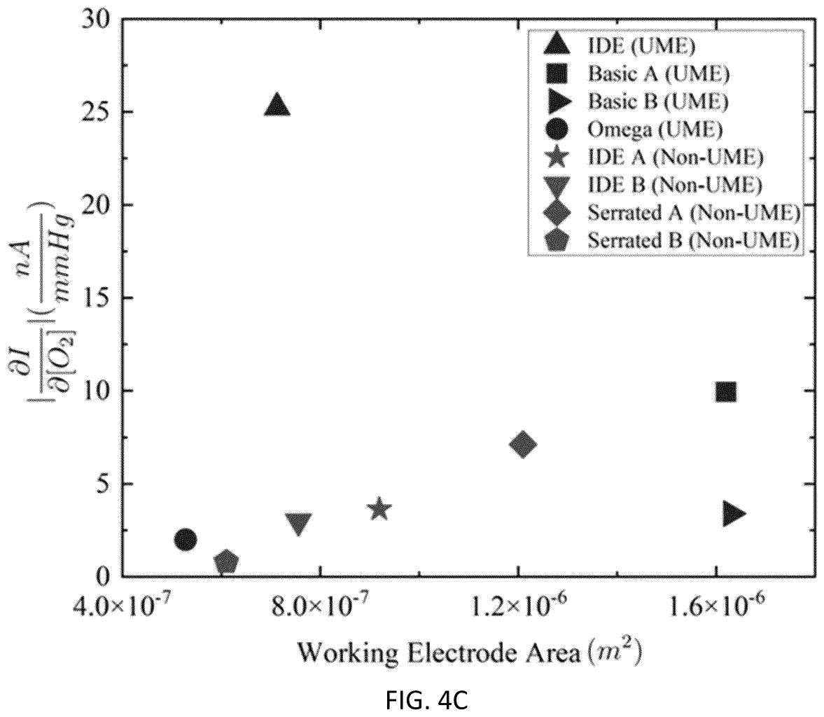

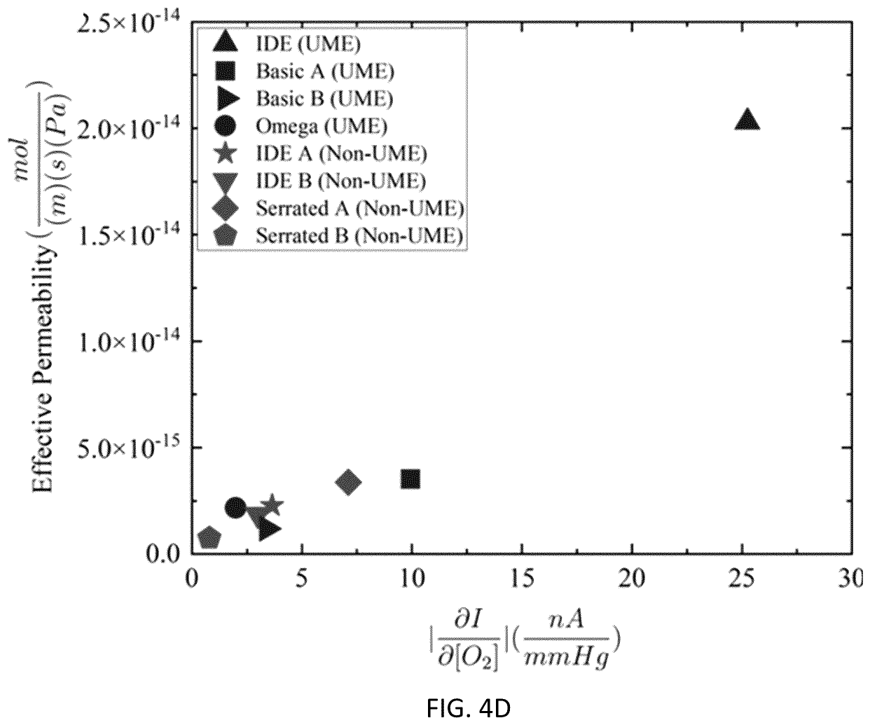

The four sensor geometries (101, 102, 103, 104) were adapted according to the specifications found in the table of FIG. 6 and were subjected to voltage sweeps from 0V to −1V across a range of oxygen concentrations. The changes in oxygen concentration were chosen to reflect the typical range of oxygen levels in cerebral tissues, from 1 mmHg to 50 mmHg (Carreau, 2011; Dings, 1998; Parrillo, 2008). The resulting I-V curves for the most responsive geometry configuration, the interdigitated geometry featuring finger widths of 20 μm and finger gaps 10 μm in length, are presented in FIG. 4A. In theory one could operate the dissolved oxygen sensor in the diffusion regime under any bias above the necessary kinetic-diffusion regime transition point; at sufficiently high voltages however, auxiliary redox reactions occur and supplement the charge transfer current from oxygen and hydrogen peroxide reduction. These reactions remove the sensor's performance from a linear regime where dissolved oxygen concentration and sensor current are directly proportional. An exponential growth in signal current is observed past a voltage of −0.7V (FIG. 4A); for this reason, the standard biasing voltage was chosen as −0.7V for the device analysis.

A linear regression analysis revealed a strong, approximately linear relationship between dissolved oxygen concentration and current response for the exemplary device 100 with an IDE geometry 104 of width 20 μm and separation of 10 μm (FIG. 4B). A slope of 0.0243 μA per mmHg of dissolved oxygen was identified for the geometry's current response. The regression analysis also yielded a strong r² value of 0.973, underscoring the high concentration dependence of the sensor's response and confirming its operation in the diffusion-limited regime.

FIG. 4C displays the working electrode area and experimental results for each geometry variation (101, 102, 103, 104) in the form of the change in current response in μA at a biasing voltage of −0.7V across a 50.3 mmHg change in dissolved oxygen concentration. According to Equation 4, the device's signal current response is linearly proportional to the working electrode area, as well as the concentration of the dissolved oxygen in solution. Several devices appear to deviate from the trend established by Equation 3, and at this point it becomes necessary to introduce a novel definition for microelectrode sensing structures: the ultramicroelectrode. Ultramicroelectrodes (UME's) have been widely defined by the electrochemical community as being electrodes that possess at least one dimension that is less than 25 μm in size (Heinze, 1993; Wang, 2006). Upon entering the UME regime, previous rules of thumb regarding electrode design no longer apply such as increasing working electrode area is no longer directly correlated with an increase in signal response. While the standard microelectrode geometries holding all dimensions above 25 μm evince a clear working electrode area dependence, the UME geometries fail to abide by the same correlation (FIG. 4C).

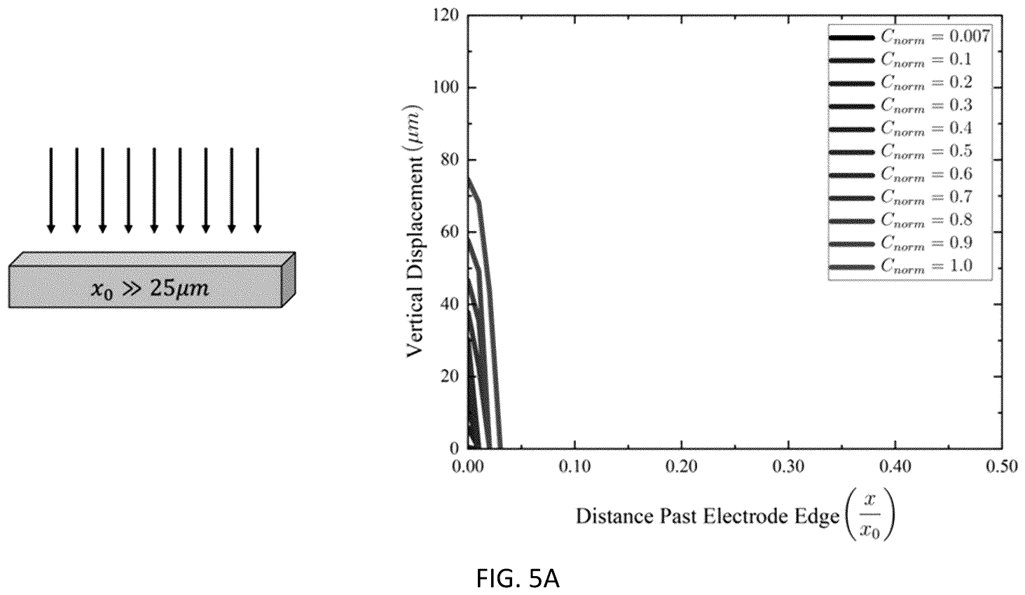

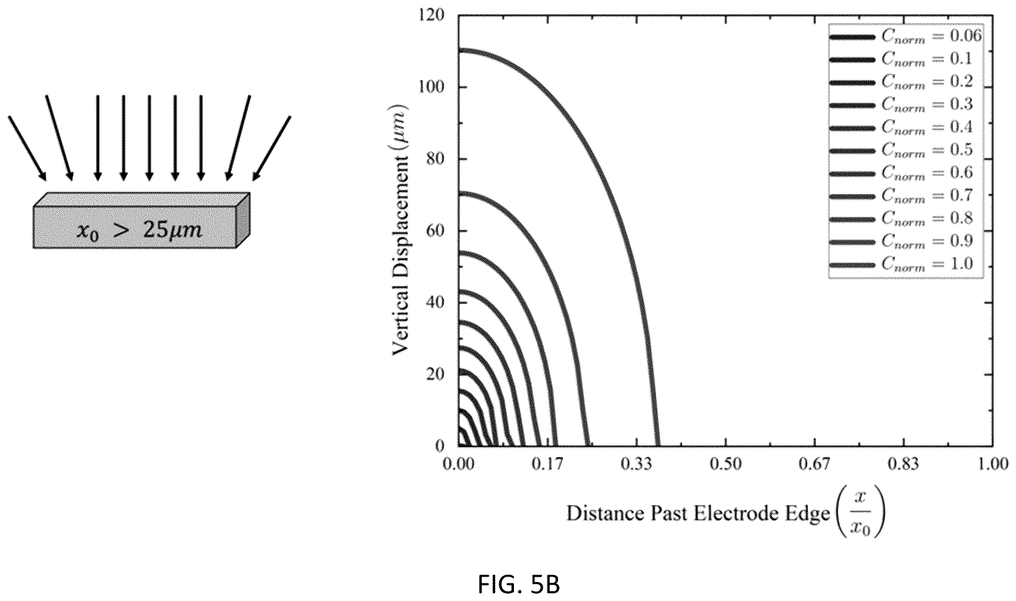

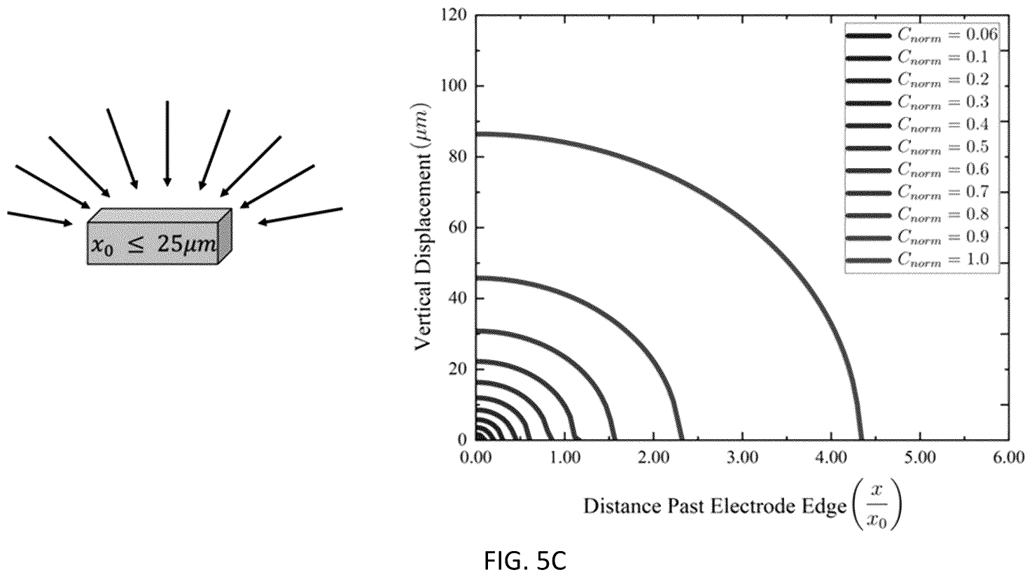

The primary reason for UME's intriguing behavior is the role of edge effects in the diffusion kinetics of the system. When operating in a diffusion-limited regime, microelectrode systems' diffusion kinetics are dominated by planar diffusion. Planar diffusion refers to a diffusion vector that is strictly orthogonal to the surface of the electrode, with an analyte concentration profile that returns to the bulk concentration outside the region directly above the electrode's surface (FIG. 5A). The surface of the electrode is assumed to be a point of zero concentration. The concentration as a function of radial distance from the electrode surface may be expressed analytically as:

$$C ( x , t ) = {C}_{0} ( 1 - \frac{{x}_{0}}{x} erf \{ \frac{( x - {x}_{0} )}{2 {( D t )}^{0.5}} \} ) \qquad ( 6 )$$

where C(x, t) is the reduced analyte concentration at a radial distance x from the electrode surface at time t after reduction initiation, C₀ is the bulk concentration, x₀ is the characteristic dimension of the working electrode, and D is the diffusivity (Bard, 2001).

As at least one dimension of the microelectrode approaches the UME regime (FIG. 5B), the diffusion takes on an additional radial component, extending the affected concentration profile beyond the electrode area [18-20]. Once the electrode enters the UME regime (FIG. 5C), the radial diffusion component begins to dominate, and the concentration profiles become hemispherical, extending the effective diffusion region far beyond the surface of the microelectrode itself (Bard, 2001; Heinze, 1993; Wang, 2006).

An increase in the effective concentration profile area gives rise to significant current densities despite low working electrode areas, all owing to a greater diffusion “collection area” (Bard, 2001; Heinze, 1993; Wang, 2006). These larger “collection areas” establish a higher sensitivity to changes in analyte concentration for ultramicroelectrode devices. While the total current may be lower than larger electrode designs, the change in signal current is much greater, boosting the robustness of microelectrode systems (Bard, 2001; McLaughlin, 2001; Wang, 2006). One critical repercussion of UME's strong signal response is that the sum of sensing currents over an array of UME's is larger than that of a single electrode of surface area equivalent to that of the UME array (McLaughlin, 2001). Consequently, when designing electrode-based electrochemical sensing systems, it is preferred to employ an array of UME's over singular, larger electrodes.

Referring to the ultramicroelectrodes' results in the table of FIG. 6, the interdigitated electrode design presented a significant signal change of 1.27 μA in response to the 50.3 mmHg change in dissolved oxygen concentration. The concentration profile behavior described by Equation 6 and illustrated in FIG. 5 was determined to be one of the key reasons behind the design's high sensitivity. The geometry's larger concentration profile supported a far higher current response when compared to non-UME designs.

The second largest signal change from the UME device group was a 0.5 μA change from the basic electrode geometry 103 with a width of 20 μm and a separation of 10 μm, nearly a factor of half less than the IDE's signal response. The inter-digitated electrode 104 array's current response indicates that the geometry itself presents an advantage over other UME designs. When the counter and working electrodes are sufficiently close together, the products at the counter electrode may diffuse back to the working electrode for re-reduction, establishing a diffusion feedback loop (Niwa, 1995; Wang, 2006). Consequently, electrode separation plays a critical role in the electrochemical sensor design, as observed in the difference in sensor performance between the two basic electrode geometries 103 (FIG. 6). Tripling the electrode separation length corresponded to a nearly proportional decrease in current response by a factor of 2.92, despite similar working and counter electrode areas.

While the diffusion feedback loop is characteristic of any electrode design with sufficiently small electrode spacing, the IDE geometry's 104 array of interdigitated working and counter electrodes poses an advantage in collection efficiency. As the oxygen is produced at one counter electrode, it diffuses to the next working electrode in the array and is then reduced again; in other words, the collection efficiency approaches unity for IDE designs (Aoki, 1988; Niwa, 1995). Such a diffusion feedback loop leads to more robust and sensitive sensing, as even low changes in dissolved oxygen concentration will be amplified by the diffusion. When compared to designs of similar dimensions in the microelectrode group, the IDE's amplification advantage becomes clear where the response from an IDE electrode 104 of width 40 μm and separation 20 μm was larger than the serrated electrode 101 of width 30 μm and separation 10 μm design by 375%, while presenting a 23% larger area.

Using Equation 5, the effective permeability for each geometry was calculated and plotted against the geometry's respective current response (FIG. 4D). Owing to its large current response and radially dominated diffusion kinetics, the ultramicroelectrode IDE geometry 104 presented the largest effective permeability.

### CONCLUSIONS

Dissolved oxygen concentration is a key parameter in monitoring processes for a wide array of industries and fields, and the optimized design of microelectrode-based oxygen sensors is critical for effective clinical applications.

The monitoring of dissolved oxygen is a key parameter in many fields, namely the treatment and monitoring of various cerebral traumas. Leveraging complementary metal oxide semiconductor (CMOS) manufacturing techniques and biocompatible electrolyte polymers, electrochemical sensors hold the potential for compact, simple, and scalable point-of-care dissolved oxygen sensors. A discussion of guiding microelectrode design principles based on a characterization comparison study conducted across multiple Clark sensors with varying microelectrode geometries was presented herein. Geometries were covered with a biocompatible Nafion polymer membrane and included variations of the classic interdigitated microelectrode array in addition to a circular microelectrode array variation. Polarographic sweeps were conducted on various microelectrode topologies while monitoring devices' sensing current responses across a 50.3 mmHg change in dissolved oxygen in deionized aqueous solution. It was observed that half of the devices demonstrated expected sensor behavior with current responses that scaled with electrode area, while four ultramicroelectrode designs presented a greater dependence on electrode spacing and electrode topology. Ultimately, ultramicroelectrodes possessing short electrode spacings and the interdigitated-electrode topology were shown to produce the greatest sensing current response.

The analysis presented here investigates the sensing performance of eight exemplary variations on four exemplary microelectrode topologies against changes in dissolved oxygen in solution. Variations of the archetypal inter-digitated electrode structure in addition to a circular electrode structure were investigated. In order to characterize the performance of each microelectrode geometry, the microelectrodes were covered with Nafion membranes, immersed in an aqueous solution containing dissolved oxygen, and subjected to voltage sweeps. Current data was collected and the changes in steady-state, diffusion-limited current across a 50.3 mmHg change in oxygen concentrations were calculated for each geometry. The observed current responses agreed well with electrochemical electrode theory and pointed to the advantages ultramicroelectrodes pose over standard microelectrode designs. While standard microelectrode designs' current responses directly followed their working electrode areas, the ultramicroelectrodes presented a stronger dependence on electrode topology and the diffusion feedback loop established by tight electrode spacing. In the final analysis, the ultramicroelectrode variation of the interdigitated electrode topology was the most sensitive geometry, generating an unexpected result of an approximately 8-fold increase in sensing current response when compared to standard IDE geometries. The ultramicroelectrode variety employed high collection efficiencies along with larger diffusion collection areas to produce improved sensing sensitivity.

The following publications are each hereby incorporated herein by reference in their entirety:
Aoki, K., M. Morita, O. Niwa, H. Tabei. (1988). Journal of Electroanalytical Chemistry 256: 269-282.
Bard, A. J., L. R. Faulkner. (2001). Electrochemical Methods 2: 168-632.
Carreau, A., B. E. Hafny-Rahbi, A. Matejuk, C. Grillon, C. Kieda (2011). Journal of Cellular and Molecular Medicine 15(6): 1239-1253.
Chue, C., C. J. Leo, W. P. Chan, M. R. B. Damalerio, M. Cheng, J. H. Cheong, Y. Gao (2015).
2015 IEEE International Conference on Electron Devices and Solid-State Circuits: 325-328.
Dings, J., J. Meixensberger, A. Jager, K. Roosen (1998). Neurosurgery 43(5): 1082-1094.
Hargreaves, J. A., C. S. Tucker (2002). Southern Regional Aquaculture Center.
Heinze, J. (1993). Angewandte Chemie International Edition English 32: 1268-1288.
Holenda, B., E. Domokos, A. Redey, J. Fazakas (2008). Computers and Chemical Engineering 32(6): 1270-1278.
Kim, T., T. Jurng, U. Chung, S. Hong (2000). Sensors and Actuators B 72: 11-20.
Li, C., C. H. Ahn, L. A. Shutter, R. K. Narayan. (2009). Biosensors and Bioelectronics 25(1): 173-178.
McLaughlin, G. W. (2001). Ph.D. thesis, Stanford University.
McLaughlin, G. W., K. Braden, B. Franc, G. T. A. Kovacs. (2002). Sensors and Actuators B 83: 138-148.
Niwa, O. (1995). Electroanalysis 7 (7): 606-613.
Parrillo, J. E., R. P. Dellinger (2008). Critical Care Medicine: Principles of Diagnosis and Management in the Adult 3: 1309-1328.
Wang, J. (2006). Analytical Electrochemistry 3: 149-163.
Wiranto, G., S. Widodo, I. D. P. Hermida, R. V. Manurung, G. Sugandi, Z. Arifin, Wiendartun (2018). Materials Science Forum 917: 59-63.
Xiao, Z., J. Wang, C. Yi, Z. Wang, L. Wang (2020). Complexity 2020.
U.S. patent application Ser. No. 15/975,067, “Multiplexed Implantable Sensor Probe”.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. An oxygen sensor device, comprising:
a Clark-type sensor electrode comprising thin-film electrode leads overlaid with a solid-state proton conductive matrix (PCM) wherein at least one dimension of the electrode is less than 25 μm.

2. The device of claim 1, wherein the electrode comprises an ultramicroelectrode.

3. The device of claim 1, wherein the Clark-type sensor electrode includes a working electrode.

4. The device of claim 1, wherein the Clark-type sensor electrode includes a counter electrode.

5. The device of claim 1, wherein the Clark-type sensor electrode includes a reference electrode.

6. The device of claim 1, further comprising titanium/gold, titanium/platinum, chromium/gold, nickel/gold or silver/silver-chloride electrodes overlaid with a PCM constructed from Nafion.

7. The device of claim 1, wherein the electrode comprises a serrated geometry.

8. The device of claim 1, wherein the electrode comprises an omega geometry.

9. The device of claim 1, wherein the electrode comprises a basic geometry.

10. The device of claim 1, wherein the electrode comprises an interdigitated (IDE) geometry.

11. The device of claim 1, wherein the device is configured to measure dissolved oxygen concentration.

12. The device of claim 1, wherein the device is configured to measure brain tissue oxygen (P_{BT}O₂).

13. An oxygen sensor system, comprising:
an oxygen sensor device as described in claim 1;
a sensing window above the electrode;
at least one contact pad; and
at least one lead line electrically connecting the at least one contact pad to the oxygen sensor electrode.

14. The system of claim 13, further comprising a protective layer overlaying the PCM.

15. The system of claim 14, wherein the protective layer comprises polydimethylsiloxane (PDMS), Polyetheretherketone (PEEK), Polytetrafluoroethylene (PTFE), Polypropylene (PP), Polystyrene (PS), Polyurethane, Polycarbonate (PC), Polyethylene, terephthalate (PET), Polymethyl methacrylate (PMMA), polymide, or Parylene-C.

16. The system of claim 13, further comprising an adhesion promoting compound.

17. The system of claim 16, wherein the adhesion promoting compound comprises polyvinylpyrollidone (PVP) or SU-8.

18. The system of claim 13, further comprising a substrate.

19. The system of claim 18, wherein the substrate comprises Kapton, glass, ceramic, silicon, or dielectric on silicon.

20. An oxygen measurement method, comprising:
providing an oxygen sensor including an electrode;
applying a voltage equal to or less than the electrochemical reduction potential of oxygen and hydrogen peroxide to the electrode;
conducting a polarographic voltage sweep;
generating a sensing current of 1 nano-amp or larger; and
calculating dissolved oxygen concentration based on the sensing current.
